(19) United States
(12) Patent Application Publication
(10) Pub. No.: US 2018/0243110 A1
(43) Pub. Date: Aug. 30, 2018

(54) ROBOTIC APPARATUS WITH AN ACTUATOR FORMED BY FIBERS

(71) Applicant: Intel Corporation, Santa Clara, CA (US)

(72) Inventors: JEREMY PARRA, Beaverton, OR (US); STEPHANIE WALKER, Albany, OR (US)

(21) Appl. No.: 15/443,806

(22) Filed: Feb. 27, 2017

(51) Int. Cl. A61F 2/68; H02N 2/06; H01L 41/113; A61F 2/54; A61F 5/01; A61H 1/02

(52) U.S. Cl. CPC A61F 2/68; H02N 2/06; H01L 41/1132; A61F 2002/704; A61F 5/0118; A61H 1/0274; A61F 2/54

(57) ABSTRACT

Embodiments of the present disclosure provide techniques and configurations for a robotic apparatus with an actuator formed by multiple fibers, in accordance with some embodiments. In some instances, the robotic apparatus may include an actuator to cause a motion of a component of a robot. The actuator may include at least one fiber that may comprise a conductive pattern. The conductive pattern may be embedded in a sheet of elastic material formed into a layered structure. The fiber may expand or contract in response to an application of a voltage signal to the conductive pattern, to cause the motion of the component of the robot. The fiber may comprise multiple fibers combined in a bundle, to form the actuator. The layered structure may comprise a roll-like shape that may be free of hollow spaces. In embodiments, the robot may comprise the robotic apparatus. Other embodiments may be described and/or claimed.

### FIELD

Embodiments of the present disclosure generally relate to the fields of robotic apparatuses, and more particularly, to actuators for wearable robotic devices.

### BACKGROUND

Wearable robotic devices may employ dielectric elastomer actuators (DEA) that use electrostatic attraction to facilitate motion. Typically, a flat elastomeric sheet may be coated on both sides with a conductive material, such as carbon grease. Electrodes may be attached to each side of the conductive material and connected to the positive or negative side of a voltage source. When the voltage source is turned on, the electrostatic attraction created from the two conductive layers may bring those layers closer together, squeezing the elastomer and simultaneously expanding the elastomer in a perpendicular direction.

However, currently used dielectric elastomer actuators may normally require high voltages (>1 kV) in order to actuate, which may not be appropriate for use on the human body. Further, existing elastomer actuators may not be able to provide a higher force application under a lower applied voltage. Also, existing elastomer actuators may not be able to provide a precise motor control on an extremity (like a user's hand).

### BRIEF DESCRIPTION OF THE DRAWINGS

Embodiments will be readily understood by the following detailed description in conjunction with the accompanying drawings. To facilitate this description, like reference numerals designate like structural elements. Embodiments are illustrated by way of example and not by way of limitation in the figures of the accompanying drawings.

FIG. 1 is a diagram illustrating an example robotic apparatus with an actuator, in accordance with some embodiments.

FIG. 2 is an example diagram illustrating some components of the robotic apparatus of FIG. 1, in accordance with some embodiments.

FIGS. 3-5 illustrate example configurations of the actuator of the robotic apparatus of FIG. 1 in different stages of assembly, in accordance with some embodiments.

FIG. 6 illustrates an example actuator for a robotic apparatus of FIG. 1, formed by multiple fibers, in accordance with some embodiments.

FIG. 7 is an example process flow diagram for providing an actuator for a robotic apparatus, in accordance with some embodiments.

FIG. 8 is an example process flow diagram for operating an actuator of a robotic apparatus, in accordance with some embodiments.

FIG. 9 illustrates an example wearable robotic apparatus with an actuator, in accordance with some embodiments.

### DETAILED DESCRIPTION

Embodiments of the present disclosure include techniques and configurations for a robotic apparatus with an actuator formed by multiple fibers, in accordance with some embodiments. In one instance, the robotic apparatus may include an actuator to cause a motion of a component of a robot. The actuator may include at least one fiber that may comprise a conductive pattern. The conductive pattern may be embedded in a sheet of elastic material formed into a layered structure. The fiber may expand or contract in response to an application of a voltage signal to the conductive pattern, to cause the motion of the component of the robot. The fiber may comprise multiple fibers combined in a bundle, to form the actuator. The layered structure may comprise a roll-like shape of the fiber that may be free of hollow spaces. In embodiments, the robot may comprise the robotic apparatus.

In the following detailed description, reference is made to the accompanying drawings that form a part hereof, wherein like numerals designate like parts throughout, and in which are shown by way of illustration embodiments in which the subject matter of the present disclosure may be practiced. It is to be understood that other embodiments may be utilized and structural or logical changes may be made without departing from the scope of the present disclosure. Therefore, the following detailed description is not to be taken in a limiting sense, and the scope of embodiments is defined by the appended claims and their equivalents.

For the purposes of the present disclosure, the phrase “A and/or B” means (A), (B), (A) or (B), or (A and B). For the purposes of the present disclosure, the phrase “A, B, and/or C” means (A), (B), (C), (A and B), (A and C), (B and C), or (A, B, and C).

The description may use perspective-based descriptions such as top/bottom, in/out, over/under, and the like. Such descriptions are merely used to facilitate the discussion and are not intended to restrict the application of embodiments described herein to any particular orientation.

The description may use the phrases “in an embodiment” or “in embodiments,” which may each refer to one or more of the same or different embodiments. Furthermore, the terms “comprising,” “including,” “having,” and the like, as used with respect to embodiments of the present disclosure, are synonymous.

The term “coupled with,” along with its derivatives, may be used herein. “Coupled” may mean one or more of the following. “Coupled” may mean that two or more elements are in direct physical, electrical, or optical contact. However, “coupled” may also mean that two or more elements indirectly contact each other, but yet still cooperate or interact with each other, and may mean that one or more other elements are coupled or connected between the elements that are said to be coupled with each other. The term “directly coupled” may mean that two or more elements are in direct contact.

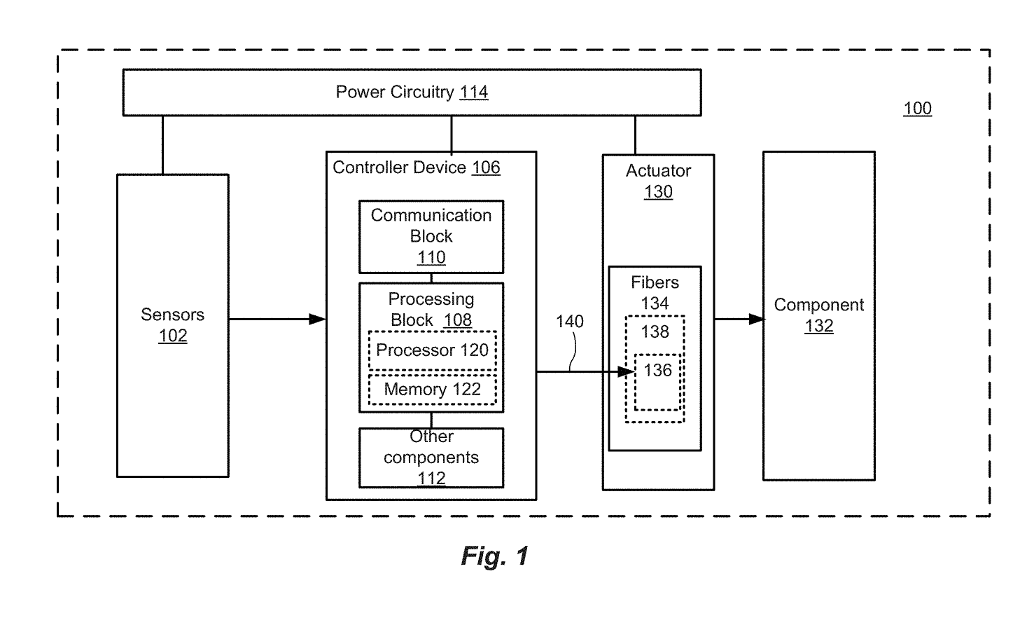

FIG. 1 is a diagram illustrating an example robotic apparatus with an actuator, in accordance with some embodiments. In embodiments, the robotic apparatus 100 may comprise a wearable robotic system that may be used for rehabilitation, assistance, and human-power augmentation. For example, the robotic apparatus 100 may comprise an upper limb or lower limb exoskeleton to improve mobility, enhance force capability, or recover motor function.

In embodiments described below in greater detail, the robotic apparatus 100 may include an actuator 130 configured to cause a motion of a movable component 132 of the robotic apparatus 100. The actuator 130 may comprise multiple fibers that may reproduce (or otherwise replicate) muscle contraction or expansion in order to cause the motion of the movable component 132.

More generally, the robotic apparatus 100 may comprise any device configured to react (e.g., move, touch, hear, or take other action or actions) in response to a sensed feedback. The sensing may include ambient light or sound sensing, pressure sensing, proximity and/or contact sensing, distance sensing, speed and/or acceleration sensing, tilt and/or orientation sensing, rotation sensing, and/or sensing of electric parameters (e.g., voltage, current, capacitance, or the like). Accordingly, one or more (e.g., a plurality of) sensors 102 may be disposed around the apparatus 100 to provide desired readings. The sensors 102 may include, but are not limited to, accelerometers, gyroscopes, proximity sensors, piezoelectric transducers, microphones, light emitting diodes (LED), cameras, lasers, LIDARs, or the like.

The apparatus may further include a controller device 106 coupled with the sensors 102, to receive sensor data readings provided by the sensors, and generate a control signal (e.g., voltage signal) 140 to provide to the actuator 130, based at least in part on sensors' readings. The controller device 106 may generate the control signal 140 in response to any type of pneumatic, hydraulic, mechanical, or electronic signals provided by the sensors 102 to the controller device 106. The controller device 106 may be electrically and/or communicatively coupled with the sensors 102, to receive and process sensor data readings and generate corresponding control signals. In embodiments, the apparatus 100 may be configured to have the controller device 106 continuously or periodically receive the sensor data readings provided by the sensors 102.

The controller device 106 may comprise, for example, a processing block 108, to process the sensor data readings, and communication block 110, to transmit a control signal, generated in response to the processing of the sensor data readings, to the actuator 130.

The processing block 108 may comprise at least a processor 120 and memory 122. The processing block 108 may include components configured to record and process the sensor data readings. The processing block 108 may provide these components through, for example, a plurality of machine-readable instructions stored in the memory 122 and executable on the processor 120.

The processor 120 may include, for example, one or more processors situated in separate components, or alternatively one or more processing cores embodied in a component (e.g., in a System-on-a-Chip (SoC) configuration), and any processor-related support circuitry (e.g., bridging interfaces, etc.). Example processors may include, but are not limited to, various microprocessors including those in the Pentium®, Xeon®, Itanium®, Celeron®, Atom®, Quark®, Core® product families, or the like.

Examples of support circuitry may include host side or input/output (I/O) side chipsets (also known as northbridge and southbridge chipsets/components) to provide an interface through which the processor 120 may interact with other system components that may be operating at different speeds, on different buses, etc. in the controller device 106. Some or all of the functionality commonly associated with the support circuitry may also be included in the same physical package as the processor.

The memory 122 may comprise random access memory (RAM) or read-only memory (ROM) in a fixed or removable format. RAM may include volatile memory configured to hold information during the operation of device 106 such as, for example, static RAM (SRAM) or Dynamic RAM (DRAM). ROM may include non-volatile (NV) memory circuitry configured based on basic input/output system (BIOS), Unified Extensible Firmware Interface (UEFI), etc. to provide instructions when the controller device 106 is activated, programmable memories such as electronic programmable ROMs (erasable programmable read-only memory), Flash, etc. Other fixed/removable memory may include, but is not limited to, electronic memories such as solid state flash memory, removable memory cards or sticks, etc.

The communication block 110 may be communicatively coupled with actuator 130 and/or an external device (not shown), and may include one or more radios capable of transmitting and receiving signals using various suitable wireless communications techniques. Some example wireless networks include (but are not limited to) wireless local area networks (WLANs) or wireless personal area networks (WPANs). In some specific non-limiting examples, the communication block 110 may comport with the Institute of Electrical and Electronics Engineers (IEEE) 802.11 standard (e.g., Wi-Fi), a Bluetooth®, ZigBee®, near-field communication, or any other suitable wireless communication standard. In some embodiments, the communication block may be coupled with the actuator 130 and/or external device through respective wired connections, and may comprise a transmitter configured to transmit data via the wired connection.

The robotic apparatus 100 may further include a power circuitry block 114 configured to provide power supply to the components of the apparatus 100, including the controller device 106. In some embodiments, the power circuitry block 114 may be configured to power on the controller device 106 continuously, periodically, or on a “wake-up” basis, in order to save battery power. The power circuitry block 114 may include internal power sources (e.g., battery, fuel cell, etc.) and/or external power sources (e.g., power grid, electromechanical or solar generator, external fuel cell, etc.) and related circuitry configured to supply the controller device 106 with the power needed to operate.

The controller device 106 may include other components 112 that may be necessary for functioning of the apparatus 100. Other components 112 may include, for example, hardware and/or software to allow users to interact with the controller device 106 such as, for example, various input mechanisms (e.g., microphones, switches, buttons, knobs, keyboards, speakers, touch-sensitive surfaces, one or more sensors configured to capture images and/or sense proximity, distance, motion, gestures, orientation, biometric data, etc.) and various output mechanisms (e.g., speakers, displays, lighted/flashing indicators, electromechanical components for vibration, motion, etc.). The hardware in other components 112 may be incorporated within the controller device 106 and/or may be coupled to the controller device 106 via a wired or wireless communication medium.

In general, the controller device 106 may be configured to cause the actuator 130 to execute a planned sequence of command, resulting in a corresponding motion of the movable component 132, in response to feedback provided by the sensors 102. The feedback may comprise indications of unforeseen errors, which may arise from inaccuracies inherent in the robotic apparatus 100, such as tolerances, static friction in joints, mechanical compliance in linkages, electrical noise on transducer signals, and/or limitations in the precision of computation.

The movable component 132 of the robotic apparatus 100 may be configured to be movable by the actuator 130, in response to controller device 106 commands, such as a voltage signal 140. The movable component 132 may include any equipment that may be activated and caused to carry out a motion by the actuator 130. For example, in wearable robotic devices, the component 132 may include any equipment that may be worn by human operators, to supplement the function of a limb or replace such function. Such equipment may operate alongside human limbs, e.g., in orthotic or exoskeleton robotic apparatuses, or may substitute human limbs. In embodiments, the robotic apparatus 100 with the component 132 may be ambulatory, portable, or autonomous. In some embodiments related to wearable robotics, the movable component 132 may need to be moved by the actuator 130 to provide an extension of the strength of a human limb, provide physical training of the limb (e.g., in a form of repetitive motions), or the like.

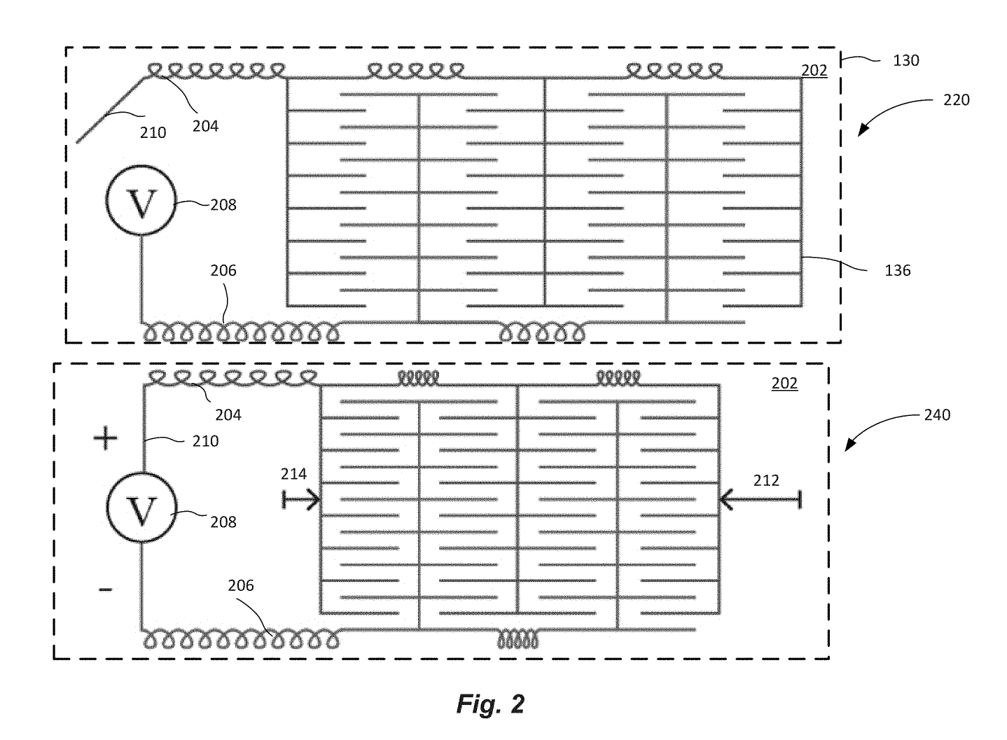

Accordingly, the actuator 130 of the robotic apparatus 100 may have the ability to imitate a muscle function of a human limb, may need to be pliable and conformable as applied to a human body, and may provide a continuous or incremental motion of the movable component 132 in response to a relatively low voltage control signal. The actuator 130 of the robotic apparatus 100 may be configured to receive the control (e.g., voltage) signal 140 from the controller device 106. As described above, the actuator 130 may comprise one or more fibers 134, which may reproduce or otherwise imitate human muscle contraction or expansion in order to cause the motion of the movable component 132. For example, one or more fibers 134 of the actuator 130 may include a conductive pattern 136 embedded in elastic material 138 in a layered structure, and may expand or contract in response to an application of the control signal 140 to the conductive pattern 136. Some embodiments of the actuator 130 are described in greater detail in reference to FIGS. 2-7.

FIG. 2 is an example diagram illustrating some components of the robotic apparatus of FIG. 1, in accordance with some embodiments. More specifically, FIG. 2 illustrates some aspects of the conductive pattern 136 of the actuator 130 of FIG. 1. For ease of understanding, like elements of FIG. 1 and subsequent figures are indicated by like numerals. In embodiments, the actuator 130 may include conductive material 202 forming the conductive pattern 136. In embodiments shown in FIG. 2, the conductive pattern 136 may comprise multiple parallel plate capacitors connected in a comb-like fashion. In some embodiments, the conductive pattern 138 may comprise other shapes, such as a zigzag pattern, a coaxial pattern, or a wave pattern. These and other types of patterns may be used as long as there are interlocking portions or concentric portions with opposite charges (attraction) or same charges (repulsion). As shown in view 220, the conductive pattern 136 may comprise electrodes 204 and 206. A control signal from the controller device 106 (shown in FIG. 1), schematically indicated as voltage 208, may be applied to the electrodes 204 and 206 in response to a closure of a switch 210. The voltage signal applied to the electrodes 204 and 206 may have the same polarity. In such case, the conductive pattern 136 comprising the conductive material 202 may expand.

In some embodiments, the voltage signal applied to one of the electrodes 204 or 206 may have different (e.g., opposite) polarity than the voltage signal applied to another one of the electrodes 204 or 206. As shown in view 240, when the voltage signals applied to electrodes 204 and 206 (e.g., switch 210 is closed) have different polarities, the pattern 136 comprising the conductive material 202 may contract, as indicated by arrows 212 and 214. The contraction (or expansion) force produced by the parallel plate capacitors comprising the conductive pattern 136 may be calculated as follows:

$$F = \frac{- 1}{2} \frac{η t {ϵ}_{0} {ϵ}_{r} {V}^{2}}{{d}^{2}} ,$$

where V is applied electric potential (voltage signal), εᵣᵣ is relative permittivity of dielectric, e₀ is permittivity of free space between the plates (e.g., 8.85 pF/m), n is total number of fingers on both sides of electrodes, t is thickness in the out-of-plane direction of the electrodes, and d is gap between electrodes.

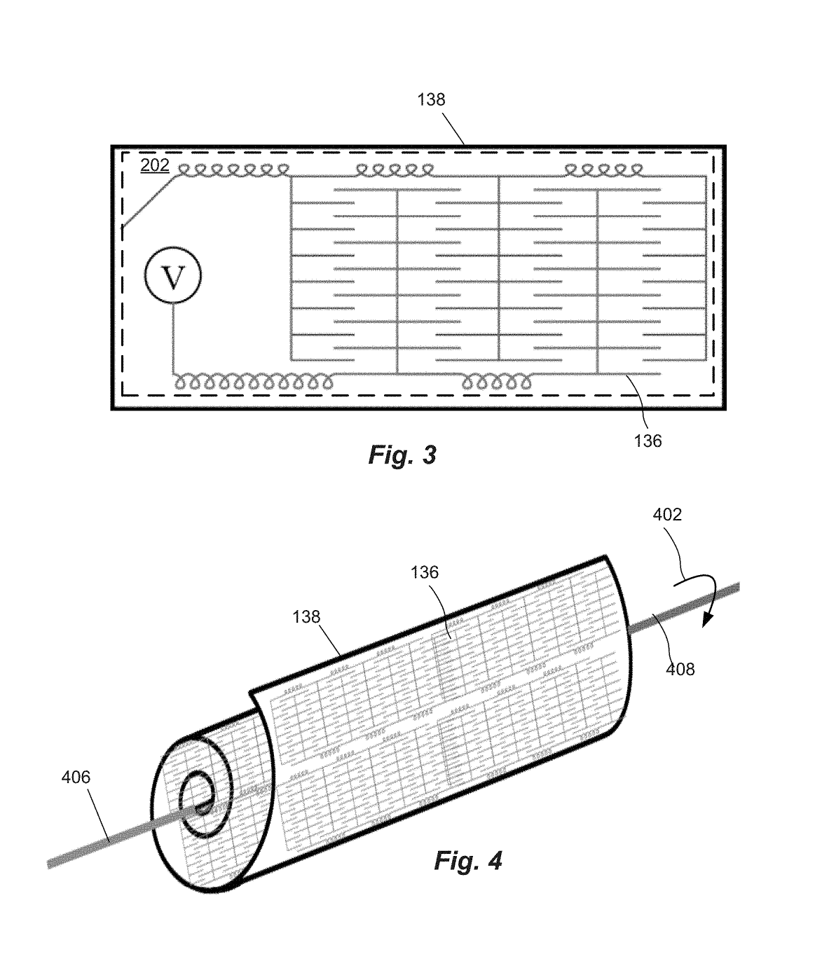

FIGS. 3-5 illustrate example configurations of the actuator of the robotic apparatus of FIG. 1 in different stages of assembly, in accordance with some embodiments.

FIG. 3 illustrates an example actuator for a robotic apparatus of FIG. 1, with a conductive pattern embedded in elastic material, in accordance with some embodiments. As described in reference to FIG. 2, the conductive pattern 136 may comprise a comb-like shape. In some embodiments, the conductive pattern 136 may comprise other shapes, such as a zigzag pattern, a coaxial pattern, or a wave pattern. In embodiments the conductive material 202 comprising the conductive pattern 136 may be embedded in a sheet of elastic material 138. In embodiments, the elastic material may comprise an elastomer. The sheet of elastic material 138 with embedded conductive pattern 136 may have a substantially flat shape, susceptible to manipulation, such as rolling, folding, or the like.

FIG. 4 illustrates an example actuator for a robotic apparatus of FIG. 1, with a conductive pattern embedded in elastic material formed into a layered structure, in accordance with some embodiments. As shown, the sheet of elastic material 138 with embedded conductive pattern 136 may be manipulated into a layered structure. For example, as indicated by arrow 402 in FIG. 4, the sheet of elastic material 138 may be rolled into a roll-like shape. In another example, the sheet of elastic material 138 may be folded into a multi-layered folded structure. Components 406 and 408 indicate respective electrodes of the conductive pattern 136, to which a control voltage signal may be applied.

FIG. 5 illustrates an example actuator for a robotic apparatus of FIG. 1, with a conductive pattern embedded in elastic material in a layered structure, forming a fiber, in accordance with some embodiments. As shown, the actuator with the layered structure formed as shown in FIG. 3, may comprise a roll-like shape, and form a wire-shaped or roll-shaped fiber 502. In embodiments, the fiber 502 may be free of hollow spaces. For example, the thickness T of the roll comprising the fiber 502 may be below 1 mm.

As described above, the fiber 502 may be configured to imitate or reproduce the action of a human muscle fiber, such as to contract or expand. For example, if the voltage applied to the electrodes 406 and 408 is of the same polarity, the fiber 502 may expand. If the voltage applied to the electrodes 406 and 408 has opposite polarities, the fiber 502 may contract.

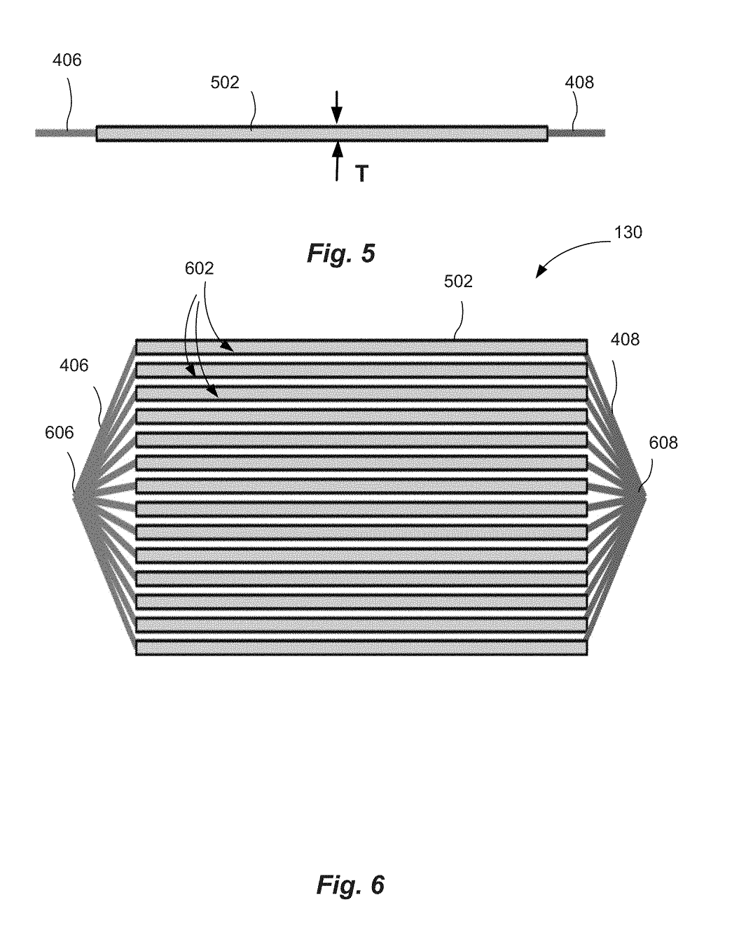

FIG. 6 illustrates an example actuator for a robotic apparatus of FIG. 1, formed by multiple fibers, in accordance with some embodiments. As shown, the actuator 130 may include multiple fibers 502 combined into a bundle of fibers 602, wherein the fibers may be connected in parallel. The electrodes 406 and 408 of the fibers 502 may be connected together to form contacts 606 and 608 respectively, as shown. Control voltage may be applied to the contacts 606 and 608, to cause the actuator 130 to contract or expand, depending on the polarity of voltage applied to the contacts 606 and 608.

The embodiments described in reference to FIGS. 1-6 may provide the following advantages compared to conventional solutions. The actuator 130, comprised of the fibers 502 forming the bundle 602 as shown in FIG. 6, may provide more force at a lower voltage due to the small distances between electrodes and layering of the conductive patterns in respective fibers of the actuator. Further, when multiple fibers are bundled into the bundle 602, the actuator 130 may become more robust. In other words, a breakage of one or even a few fibers may not affect the overall performance of the actuator 130. Also, miniaturization of the described actuator embodiments may be possible based on existing industrial technologies. Other materials may be combined to produce a suite of fiber functionalities for the actuator 130.

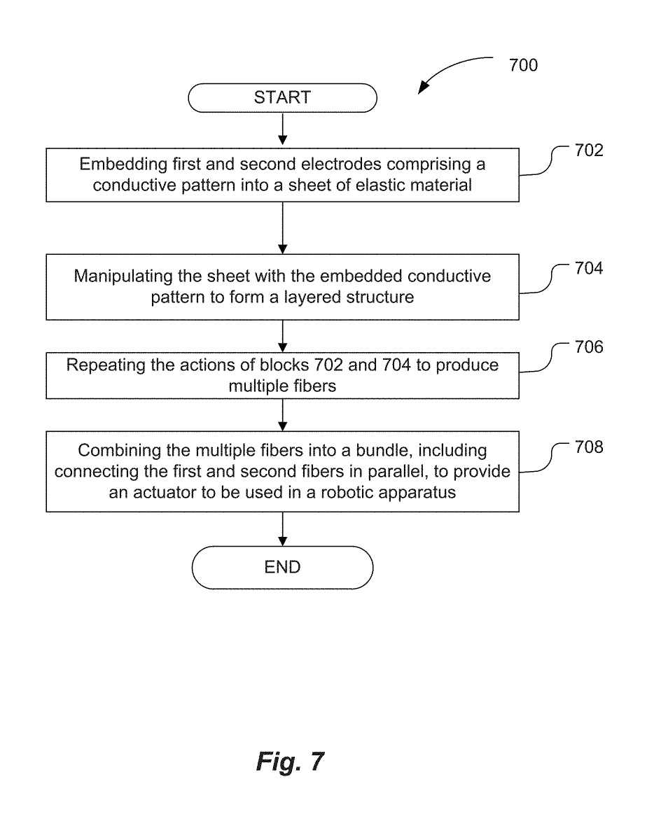

FIG. 7 is an example process flow diagram for providing an actuator for a robotic apparatus, in accordance with some embodiments. The process 700 may comport with some of the apparatus embodiments described in reference to FIGS. 1-6.

The process 700 may begin at block 702 and include embedding first and second electrodes comprising a conductive pattern into a sheet of elastic material. In embodiments, the elastic material may comprise an elastomer, and the conductive pattern may comprise a comb pattern, a wave-like pattern, a coaxial pattern, or a zigzag pattern.

At block 704, the process 700 may include manipulating the sheet with the embedded conductive pattern to form a layered structure, to provide a fiber that may expand or contract in response to applying a voltage signal to the first and second electrodes. The resulting layered structure may form a roll or folded structure, and may be free of hollow spaces.

At block 706, the process 700 may include repeating the actions of blocks 702 and 704 to produce multiple fibers.

At block 708, the process 700 may include combining the multiple fibers into a bundle, including connecting the first and second fibers in parallel. The resulting bundle may comprise an actuator to be used in a robotic apparatus, such as a wearable robotic device.

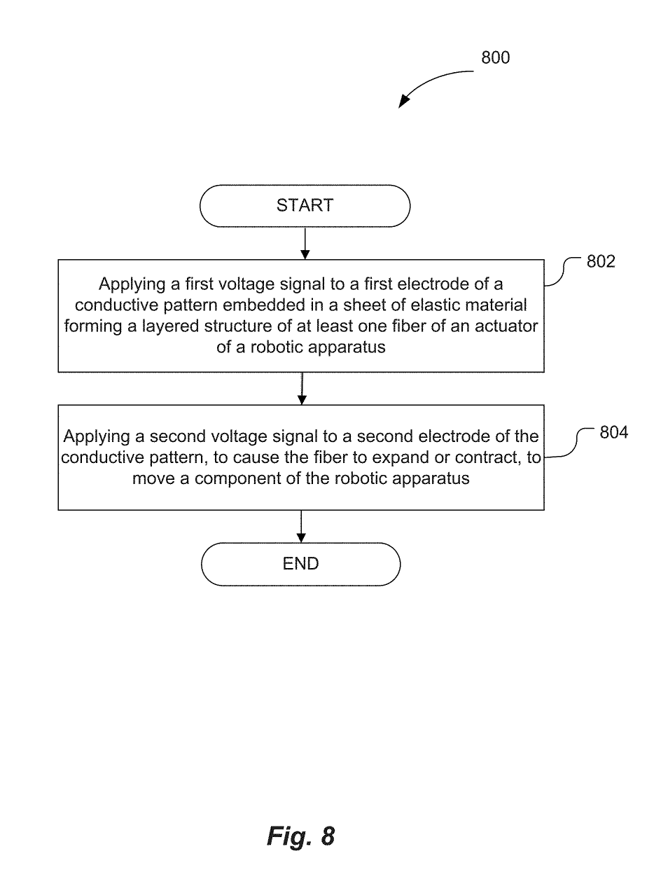

FIG. 8 is an example process flow diagram for operating an actuator of a robotic apparatus, in accordance with some embodiments. The process 800 may be performed by the controller 106 of the apparatus 100 of FIG. 1. In alternate embodiments, the process 800 may be practiced with more or fewer operations, or a different order of the operations.

The process 800 may begin at block 802 and include applying a first voltage signal to a first electrode of a conductive pattern embedded in a sheet of elastic material forming a layered structure of at least one fiber of an actuator of a robotic apparatus. As discussed, the actuator may include one or more fibers comprising a layered structure of the conductive pattern embedded in the elastic material.

At block 804, the process 800 may include applying a second voltage signal to a second electrode of the conductive pattern, wherein applying the first and second voltages to the first and second electrodes may cause the fiber to expand or contract, to move a component of the robotic apparatus.

In some embodiments, applying the first and second voltage signals may include providing the first voltage signal of a same polarity as the second voltage signal. Accordingly, the fiber, and consequently the actuator, may expand in response to applying the voltage signals to the electrodes of the conductive pattern.

In some embodiments, applying the first and second voltage signals may include providing the first voltage signal and second voltage signals of opposite polarities. Accordingly, the fiber, and consequently the actuator, may contract in response to applying the voltage signals to the electrodes of the conductive pattern.

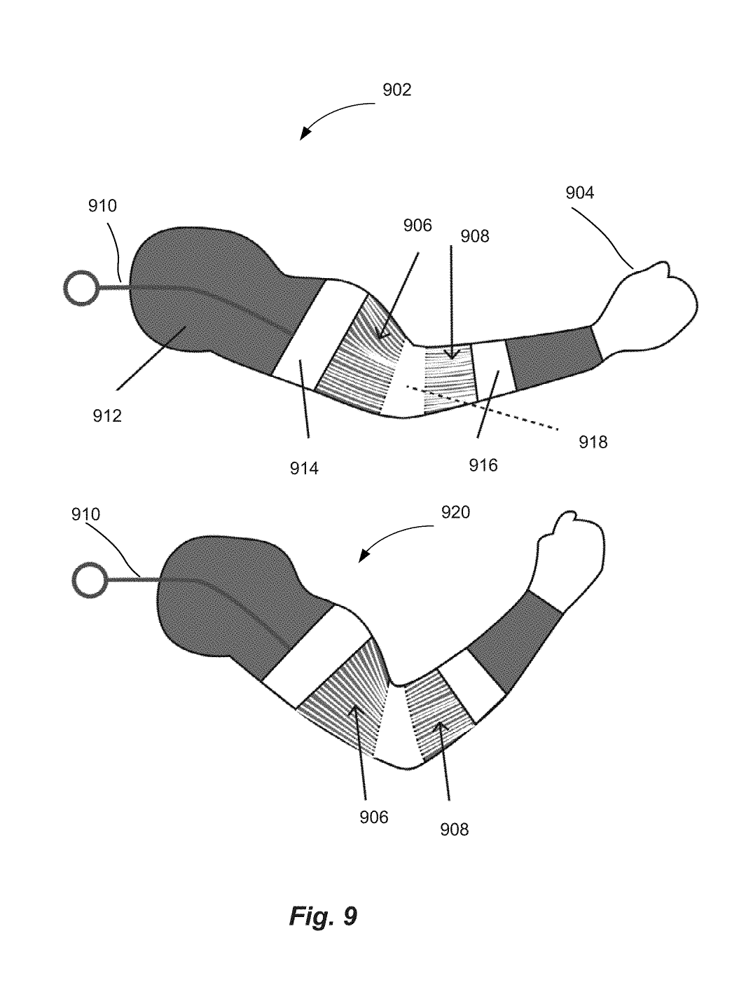

FIG. 9 illustrates an example wearable robotic apparatus with an actuator, in accordance with some embodiments. More specifically, view 902 illustrates the robotic apparatus in a default position on a human joint 904 (e.g., control voltage off), and view 920 illustrates the robotic apparatus in a contracted position on the human joint 904 (e.g. control voltage on, opposite charge). As shown, the actuator of the robotic apparatus in accordance with some embodiments described herein may include fiber bundles 906, 908 (shown in contracted state in view 920). Connector 910 may provide connections to a control system (e.g. controller device 106 of FIG. 1, not shown in FIG. 9). The apparatus may include a sleeve 912 provided under fibers for comfort. The sleeve 912 may also hold connections to controls (e.g., controller device 106). The apparatus may further include elastic anchor bands 914, 916, 918. As shown, the fibers of the bundles 906, 908 may slip through elastic anchor band 918 and may be controlled and/or held by the elastic anchor band 918.

The following paragraphs describe examples of various embodiments.

Example 1 may be a robotic apparatus, comprising: an actuator to cause a motion of a component of a robot, wherein the actuator includes at least one fiber that comprises a conductive pattern, wherein the conductive pattern is embedded in a sheet of elastic material formed into a layered structure, wherein the at least one fiber is to expand or contract in response to an application of a voltage signal to the conductive pattern, to cause the motion of the component of the robot.

Example 2 may include the robotic apparatus of example 1, further comprising at least one sensor coupled with the component to generate a sensor signal indicative of the motion of the component.

Example 3 may include the robotic apparatus of example 2, further comprising a controller device coupled with the at least one sensor and the actuator, to generate the voltage signal, based at least in part on the sensor signal, and to provide the voltage signal to the actuator.

Example 4 may include the robotic apparatus of example 1, wherein the elastic material comprises elastomer.

Example 5 may include the robotic apparatus of example 1, wherein the conductive pattern comprises one of: a comb pattern, a zigzag pattern, a coaxial pattern, or a wave pattern.

Example 6 may include the robotic apparatus of example 1, wherein the conductive pattern comprises first and second electrodes, wherein the voltage signal applied to the conductive pattern includes a first voltage signal applied to the first electrode, and a second voltage signal applied to the second electrode.

Example 7 may include the robotic apparatus of example 6, wherein the first voltage signal has a same polarity as the second voltage signal, wherein the fiber is to expand in response to the application of the voltage signal to the conductive pattern.

Example 8 may include the robotic apparatus of example 6, wherein the first voltage signal has a different polarity than the second voltage signal, wherein the fiber is to contract in response to the application of the voltage signal to the conductive pattern.

Example 9 may include the robotic apparatus of example 1, wherein the at least one fiber comprises multiple fibers combined in a bundle.

Example 10 may include the robotic apparatus of example 1, wherein the layered structure comprises a roll-like shape of the fiber that is free of hollow spaces.

Example 11 may include the robotic apparatus of example 1, wherein the robotic apparatus comprises a wearable device, wherein the actuator comprises a dielectric elastomer actuator (DEA).

Example 12 may include the robotic apparatus of any examples 1 to 11, wherein the robot comprises the robotic apparatus.

Example 13 may be a method for providing an actuator for a robotic apparatus, comprising: embedding first and second electrodes comprising a conductive pattern into a sheet of elastic material; and manipulating the sheet to form a layered structure of the conductive pattern, to provide a fiber that is to expand or contract in response to applying a voltage signal to the first and second electrodes, to actuate a motion of a component of the robotic apparatus that is to be connected with the fiber.

Example 14 may include the method of example 13, wherein the conductive pattern is a first conductive pattern, wherein the sheet of elastic material is a first sheet, wherein a fiber is a first fiber, wherein the layered structure is a first layered structure, wherein the method further comprises: embedding third and fourth electrodes comprising a second conductive pattern into a second sheet of elastic material; manipulating the second sheet to form a second layered structure of a second conductive pattern, to provide a second fiber responsive to application of the voltage signal to the third and fourth electrodes; and combining the first and second fibers, to form a bundle, including connecting the first and second fibers in parallel, wherein the bundle comprises an actuator to be used in the robotic apparatus.

Example 15 may include the method of example 14, wherein manipulating the first and second sheets to form the first and second layered structures includes providing the first and second layered structures that are free of hollow spaces.

Example 16 may include the method of example 15, further comprising: forming the conductive pattern, wherein the conductive pattern includes one of: a comb pattern, a zigzag pattern, a coaxial pattern, or a wave pattern.

Example 17 may be a method for using an actuator in a robotic apparatus, comprising: applying a first voltage signal to a first electrode of a conductive pattern embedded in a sheet of elastic material forming a layered structure of at least one fiber of an actuator of a robotic apparatus; and applying a second voltage signal to a second electrode of the conductive pattern, wherein applying the first and second voltages to the first and second electrodes causes the at least one fiber to expand or contract, to actuate a motion of a component of the robotic apparatus.

Example 18 may include the method of example 17, wherein applying the first and second voltage signals includes providing the first voltage signal of a same polarity as the second voltage signal, wherein the fiber is to expand in response to applying the first and second voltage signals to the first and second electrodes of the conductive pattern respectively.

Example 19 may include the method of any examples 17 to 18, wherein applying the first and second voltage signals includes providing the first voltage signal of a different polarity than the second voltage signal, wherein the fiber is to contract in response to applying the first and second voltage signals to the first and second electrodes of the conductive pattern respectively.

Example 20 may be a robotic system, comprising: a component; a controller coupled with the component, to control a motion of the component; and an actuator coupled with the controller and the component, to cause the motion of a component in response to a control command generated by the controller, wherein the actuator includes at least one fiber that comprises a conductive pattern, wherein the pattern is embedded in a sheet of elastic material formed into a layered structure, wherein the at least one fiber is to expand or contract in response to an application of a voltage signal to the conductive pattern, wherein the voltage signal indicates the control command generated by the controller.

Example 21 may include the robotic system of example 20, further comprising at least one sensor coupled with the component to generate a sensor signal indicative of the motion of the component, wherein the controller is to provide the control command in response to a receipt of the generated sensor signal.

Example 22 may include the robotic system of example 20, wherein the elastic material comprises elastomer, wherein the conductive pattern comprises one of: a comb shape, a zigzag shape, a coaxial shape, or a wavelike shape.

Example 23 may include the robotic system of example 20, wherein the conductive pattern comprises first and second electrodes, wherein the voltage signal applied to the conductive pattern includes a first voltage signal applied to the first electrode, and a second voltage signal applied to the second electrode.

Example 24 may include the robotic system of example 20, wherein the first voltage signal has a same polarity as the second voltage signal, wherein the fiber is to expand in response to the application of the voltage signal to the conductive pattern, or wherein the first voltage signal has a different polarity than the second voltage signal, wherein the fiber is to contract in response to the application of the voltage signal to the conductive pattern.

Example 25 may include the robotic system of any examples 20 to 24, wherein the layered structure comprises a roll-like shape of the fiber that is free of hollow spaces.

Various operations are described as multiple discrete operations in turn, in a manner that is most helpful in understanding the claimed subject matter. However, the order of description should not be construed as to imply that these operations are necessarily order dependent. Embodiments of the present disclosure may be implemented into a system using any suitable hardware and/or software to configure as desired.

Although certain embodiments have been illustrated and described herein for purposes of description, a wide variety of alternate and/or equivalent embodiments or implementations calculated to achieve the same purposes may be substituted for the embodiments shown and described without departing from the scope of the present disclosure. This application is intended to cover any adaptations or variations of the embodiments discussed herein. Therefore, it is manifestly intended that embodiments described herein be limited only by the claims and the equivalents thereof.

What is claimed is:

1. A robotic apparatus, comprising:
an actuator to cause a motion of a component of a robot, wherein the actuator includes at least one fiber that comprises a conductive pattern, wherein the conductive pattern is embedded in a sheet of elastic material formed into a layered structure, wherein the at least one fiber is to expand or contract in response to an application of a voltage signal to the conductive pattern, to cause the motion of the component of the robot.

2. The robotic apparatus of claim 1, further comprising at least one sensor coupled with the component to generate a sensor signal indicative of the motion of the component.

3. The robotic apparatus of claim 2, further comprising a controller device coupled with the at least one sensor and the actuator, to generate the voltage signal, based at least in part on the sensor signal, and to provide the voltage signal to the actuator.

4. The robotic apparatus of claim 1, wherein the elastic material comprises elastomer.

5. The robotic apparatus of claim 1, wherein the conductive pattern comprises one of: a comb pattern, a zigzag pattern, a coaxial pattern, or a wave pattern.

6. The robotic apparatus of claim 1, wherein the conductive pattern comprises first and second electrodes, wherein the voltage signal applied to the conductive pattern includes a first voltage signal applied to the first electrode, and a second voltage signal applied to the second electrode.

7. The robotic apparatus of claim 6, wherein the first voltage signal has a same polarity as the second voltage signal, wherein the fiber is to expand in response to the application of the voltage signal to the conductive pattern.

8. The robotic apparatus of claim 6, wherein the first voltage signal has a different polarity than the second voltage signal, wherein the fiber is to contract in response to the application of the voltage signal to the conductive pattern.

9. The robotic apparatus of claim 1, wherein the at least one fiber comprises multiple fibers combined in a bundle.

10. The robotic apparatus of claim 1, wherein the layered structure comprises a roll-like shape of the fiber that is free of hollow spaces.

11. The robotic apparatus of claim 1, wherein the robotic apparatus comprises a wearable device, wherein the actuator comprises a dielectric elastomer actuator (DEA).

12. The robotic apparatus of claim 1, wherein the robot comprises the robotic apparatus.

13. A method for providing an actuator for a robotic apparatus, comprising:
embedding first and second electrodes comprising a conductive pattern into a sheet of elastic material; and
manipulating the sheet to form a layered structure of the conductive pattern, to provide a fiber that is to expand or contract in response to applying a voltage signal to the first and second electrodes, to actuate a motion of a component of the robotic apparatus that is to be connected with the fiber.

14. The method of claim 13, wherein the conductive pattern is a first conductive pattern, wherein the sheet of elastic material is a first sheet, wherein a fiber is a first fiber, wherein the layered structure is a first layered structure, wherein the method further comprises:
embedding third and fourth electrodes comprising a second conductive pattern into a second sheet of elastic material;
manipulating the second sheet to form a second layered structure of a second conductive pattern, to provide a second fiber responsive to application of the voltage signal to the third and fourth electrodes; and
combining the first and second fibers, to form a bundle, including connecting the first and second fibers in parallel, wherein the bundle comprises an actuator to be used in the robotic apparatus.

15. The method of claim 14, wherein manipulating the first and second sheets to form the first and second layered structures includes providing the first and second layered structures that are free of hollow spaces.

16. The method of claim 15, further comprising: forming the conductive pattern, wherein the conductive pattern includes one of: a comb pattern, a zigzag pattern, a coaxial pattern, or a wave pattern.

17. A method, comprising:
applying a first voltage signal to a first electrode of a conductive pattern embedded in a sheet of elastic material forming a layered structure of at least one fiber of an actuator of a robotic apparatus; and
applying a second voltage signal to a second electrode of the conductive pattern, wherein applying the first and second voltages to the first and second electrodes causes the at least one fiber to expand or contract, to actuate a motion of a component of the robotic apparatus.

18. The method of claim 17, wherein applying the first and second voltage signals includes providing the first voltage signal of a same polarity as the second voltage signal, wherein the fiber is to expand in response to applying the first and second voltage signals to the first and second electrodes of the conductive pattern respectively.

19. The method of claim 17, wherein applying the first and second voltage signals includes providing the first voltage signal of a different polarity than the second voltage signal, wherein the fiber is to contract in response to applying the first and second voltage signals to the first and second electrodes of the conductive pattern respectively.

20. A robotic system, comprising:
a component;
a controller coupled with the component, to control a motion of the component; and
an actuator coupled with the controller and the component, to cause the motion of a component in response to a control command generated by the controller, wherein the actuator includes at least one fiber that comprises a conductive pattern, wherein the pattern is embedded in a sheet of elastic material formed into a layered structure, wherein the at least one fiber is to expand or contract in response to an application of a voltage signal to the conductive pattern, wherein the voltage signal indicates the control command generated by the controller.

21. The robotic system of claim 20, further comprising at least one sensor coupled with the component to generate a sensor signal indicative of the motion of the component, wherein the controller is to provide the control command in response to a receipt of the generated sensor signal.

22. The robotic system of claim 20, wherein the elastic material comprises elastomer, wherein the conductive pattern comprises one of: a comb shape, a zigzag shape, a coaxial shape, or a wavelike shape.

23. The robotic system of claim 20, wherein the conductive pattern comprises first and second electrodes, wherein the voltage signal applied to the conductive pattern includes a first voltage signal applied to the first electrode, and a second voltage signal applied to the second electrode.

24. The robotic system of claim 20, wherein the first voltage signal has a same polarity as the second voltage signal, wherein the fiber is to expand in response to the application of the voltage signal to the conductive pattern, or wherein the first voltage signal has a different polarity than the second voltage signal, wherein the fiber is to contract in response to the application of the voltage signal to the conductive pattern.

25. The robotic system of claim 20, wherein the layered structure comprises a roll-like shape of the fiber that is free of hollow spaces.
